(19) United States
(12) Patent Application Publication
(10) Pub. No.: US 2019/0082937 A1
(43) Pub. Date: Mar. 21, 2019

(54) ADHESIVE COMPOSITION, ULTRASONIC TRANSDUCER, ENDOSCOPE, AND ULTRASONIC ENDOSCOPE

(71) Applicant: OLYMPUS CORPORATION, Tokyo (JP)

(72) Inventors: Takae HAYASHI, Aizu-Wakamatsu-shi (JP); Koji KOBAYASHI, Tokyo (JP); Hiroki YOKOYAMA, Aizu-Wakamatsu-shi (JP)

(73) Assignee: OLYMPUS CORPORATION, Tokyo (JP)

(21) Appl. No.: 16/192,552

(22) Filed: Nov. 15, 2018

Related U.S. Application Data
(63) Continuation of application No. PCT/JP2017/018170, filed on May 15, 2017.

(30) Foreign Application Priority Data
May 27, 2016 (JP) 2016-106558
Nov. 14, 2016 (JP) 2016-221826

(51) Int. Cl. A61B 1/00; C09J 163/04; A61B 8/00; A61B 1/005

(52) U.S. Cl. CPC A61B 1/00071; C09J 163/04; C09J 11/04; A61B 1/005; A61B 8/4422

(57) ABSTRACT

An adhesive composition includes an epoxy resin as a main component and an inorganic zwitterion exchanger.

The application is a continuation application based on a PCT Patent Application No. PCT/JP2017/018170, filed May 15, 2017, whose priority is claimed on Japanese Patent Application No. 2016-106558, filed May 27, 2016, and Japanese Patent Application No. 2016-221826, filed Nov. 14, 2016. The content of both the PCT Application and the Japanese Applications are incorporated herein by reference.

### FIELD OF THE INVENTION

The present invention relates to an adhesive composition, an ultrasonic transducer, an endoscope, and an ultrasonic endoscope.

### Description of Related Art

In recent years, minimally invasive medical treatments in which a burden on patients is reduced have been focused on. For example, as one minimally invasive medical treatment, a treatment method using an endoscope is known.

In endoscopes, in order to maintain liquid tightness of an insertion portion, for example, a binding thread is used for fixing a side surface of a cap of a distal end portion and an outer tube covering a side surface of the insertion portion. For example, while the outer tube is externally fitted to the side surface of the cap, the binding thread is wound around the surface of the outer tube. When the outer tube is tightly bound to the side surface of the cap by the binding thread, the outer tube is fixed to the cap in a liquid-tight manner. Furthermore, in order to prevent the binding thread from being unwound, the binding thread is covered with an adhesive layer formed by curing a thermosetting adhesive.

Such an endoscope is subjected to a sterilization treatment because the endoscope is inserted into a patient's body and then used. There are various methods for a sterilization treatment. Recently, sterilization treatments using a sterilizing gas highly effective even at low temperatures are being performed increasingly often.

For example, in a gas-based sterilization treatment using hydrogen peroxide plasma, the power of chemical attack on a member constituting the endoscope is increasing. For example, adhesives for protecting the binding thread also need to have a higher resistance with respect to a sterilizing gas.

For example, in Japanese Unexamined Patent Application, First Publication No. 2014-210836, an adhesive composition having an excellent sterilization resistance with respect to hydrogen peroxide plasma sterilization and an endoscope using the same are described. The adhesive composition described in the above Application includes an ion exchanger.

An ion exchanger is a substance having a property of allowing exchange of ions of the ion exchanger itself and ions present around the ion exchanger. The ion exchanger is said to capture surrounding ions, and thus the ion exchanger is also called an ion scavenger.

In the above Application, an organic ion exchanger and an inorganic anion exchanger are disclosed as ion exchangers used in the adhesive composition.

### SUMMARY OF THE INVENTION

An adhesive composition of a first aspect of the present invention includes an epoxy resin as a main component and an inorganic zwitterion exchanger.

According to an adhesive composition of a second aspect of the present invention, in the first aspect, the inorganic zwitterion exchanger may be an inorganic compound including at least one type of metal atom selected from a group consisting of bismuth, antimony, zirconium, magnesium, and aluminum.

According to an adhesive composition of a third aspect of the present invention, in the first aspect, 0.1 parts by mass or more and 1.0 part by mass or less of the inorganic zwitterion exchanger may be added with respect to 10 parts by mass of the epoxy resin.

According to an adhesive composition of a fourth aspect of the present invention, in the first aspect, the epoxy resin may include at least one type of epoxy resin selected from a group consisting of a bisphenol A type epoxy resin, a bisphenol F type epoxy resin, and a phenol novolak type epoxy resin.

According to an adhesive composition of a fifth aspect of the present invention, in the first aspect, the adhesive composition may further include a curing agent including at least one selected from a group consisting of xylenediamine, a polyamine, a tertiary amine, and derivatives thereof.

According to an adhesive composition of a sixth aspect of the present invention, in the first aspect, the adhesive composition may further include an inorganic filler.

According to an adhesive composition of a seventh aspect of the present invention, in the sixth aspect, the inorganic filler may include at least one type of inorganic filler selected from a group consisting of alumina, zirconia, silicon nitride, silicon carbide, tungsten trioxide, diamond, sapphire, aluminum nitride, boron nitride, and magnesium oxide.

According to an adhesive composition of an eighth aspect of the present invention, in the sixth aspect, 30 parts by mass or more and 300 parts by mass or less of the inorganic filler may be included with respect to 10 parts by mass of the epoxy resin.

According to an adhesive composition of a ninth aspect of the present invention, in the sixth aspect, the inorganic filler may be spherical particles having an aspect ratio of 0 or more and less than 0.5.

An ultrasonic transducer of a tenth aspect of the present invention has an acoustic matching layer including a cured resin layer obtained by curing the adhesive composition according to the sixth aspect.

An endoscope of an eleventh aspect of the present invention includes two of constituent members and an adhesive layer obtained by curing the adhesive composition of the first aspect of the present invention. The two constituent members are bonded to each other by the adhesive composition.

An ultrasonic endoscope of a twelfth aspect of the present invention includes the ultrasonic transducer of the tenth aspect.

### BRIEF DESCRIPTION OF THE DRAWINGS

FIG. 1 is a perspective view schematically showing a schematic configuration of an endoscope according to a first embodiment of the present invention.

FIG. 2 is a cross-sectional view schematically showing an outer tube-fixing part at a distal end portion of the endoscope according to the first embodiment of the present invention.

FIG. 3 is a front view schematically showing a distal end portion of the endoscope according to the first embodiment of the present invention.

FIG. 4 is a front view schematically showing a schematic configuration of an ultrasonic endoscope according to a second embodiment of the present invention.

FIG. 5 is a cross-sectional view schematically showing a configuration of main parts of the ultrasonic endoscope according to the second embodiment of the present invention.

FIG. 6 is a cross-sectional view schematically showing a schematic configuration of an ultrasonic transducer according to a third embodiment of the present invention.

### DETAILED DESCRIPTION OF THE INVENTION

Embodiments of the present invention will be described below with reference to the appended drawings. In all of the drawings, even if embodiments are different, the same or corresponding members will be denoted with the same reference numerals and common descriptions will be omitted.

### First Embodiment

An adhesive composition and an endoscope according to a first embodiment of the present invention will be described below.

The inventors conducted extensive studies in order to further improve a sterilization resistance of an adhesive layer obtained by curing an adhesive composition in a sterilization treatment using a sterilizing gas. The inventors newly found that a sterilization resistance of the adhesive layer can be significantly improved by incorporating an inorganic zwitterion exchanger which has not been used for an adhesive for a medical instrument into an adhesive composition, and completed the present invention.

The adhesive composition of the present embodiment includes an epoxy resin and an inorganic zwitterion exchanger. Here, the inorganic zwitterion exchanger is an inorganic compound having a property of allowing exchange of surrounding anions and cations with its own ions.

At least one of an acrylic rubber and a filler may be added to the adhesive composition of the present embodiment. The adhesive composition of the present embodiment may include a curing agent.

Hereinafter, when an acrylic rubber is included in the adhesive composition of the present embodiment, the acrylic rubber and the epoxy resin are referred to as a main agent. When no acrylic rubber is included in the adhesive composition of the present embodiment the epoxy resin is referred to as a main agent.

The adhesive composition of the present embodiment is suitably used for an adhesive of a constituent member of a medical instrument for example, an endoscope, as an adhesive for a medical instrument.

The adhesive layer formed by curing the adhesive composition of the present embodiment has a favorable resistance with respect to a sterilization treatment using various sterilizing gases.

As the epoxy resin used in the adhesive composition of the present embodiment at least one type selected from the group consisting of a bisphenol A type epoxy resin, a bisphenol F type epoxy resin, and a phenol novolak type epoxy resin is more preferably included.

The epoxy resin may include the three types of a bisphenol A type epoxy resin, a bisphenol F type epoxy resin, and a phenol novolak type epoxy resin. In this case, the adhesive layer can have a higher sterilization resistance with respect to a sterilization treatment that is repeatedly performed, and higher adhesive strength can be obtained. Furthermore, in this case, the viscosity of the adhesive composition is easily adjusted.

The content of the bisphenol A type epoxy resin may be 20 parts by mass or more and 70 parts by mass or less with respect to 100 parts by mass of the main agent. The content of the bisphenol A type epoxy resin is more preferably 30 parts by mass or more and 60 parts by mass or less with respect to 100 parts by mass of the main agent.

Specific examples of the bisphenol A type epoxy resin include Adeka resin EP (registered trademark)-4100E (product name; commercially available from ADEKA), Acryset (registered trademark) BPA328 (product name; commercially available from Nippon Shokubai Co., Ltd.), and jER (registered trademark) 828 (product name; commercially available from Mitsubishi Chemical Corporation).

The content of the bisphenol F type epoxy resin may be 10 parts by mass or more and 60 parts by mass or less with respect to 100 parts by mass of the main agent. The content of the bisphenol F type epoxy resin is more preferably 30 parts by mass or more and 60 parts by mass or less with respect to 100 parts by mass of the main agent.

Specific examples of the bisphenol F type epoxy resin include Acryset (registered trademark) BPF307 (product name; commercially available from Nippon Shokubai Co., Ltd.), and jER (registered trademark) 807 (product name; commercially available from Mitsubishi Chemical Corporation).

The content of the phenol novolak type epoxy resin may be 20 parts by mass or more and 40 parts by mass or less with respect to 100 parts by mass of the main agent. The content of the phenol novolak type epoxy resin is more preferably 30 parts by mass or more and 70 parts by mass or less with respect to 100 parts by mass of the main agent.

Specific examples of the phenol novolak type epoxy resin include jER (registered trademark) 152 (product name; commercially available from Mitsubishi Chemical Corporation), and EPICLON (registered trademark) N-770 (product name; commercially available from DIC).

When an acrylic rubber is included in the main agent of the adhesive composition of the present embodiment, the acrylic rubber has a function of imparting a moisture and heat resistance to withstand a sterilization treatment particularly under high-temperature and high-pressure streams to the adhesive composition and maintaining adhesive strength more favorably.

The acrylic rubber is used by being dispersed in the above epoxy resin. An acrylic rubber in the form of a fine powder having, for example, an average particle size of 300 nm or less, may be used.

When the epoxy resin in which the acrylic rubber is dispersed is heated, a sea-island structure in which an acrylic rubber is distributed like islands in the epoxy resin is formed. Due to the sea-island structure of the acrylic rubber, adhesive characteristics such as sterilization resistance are likely to be exhibited under high-temperature and high-humidity conditions.

Generally, it is said that formation of the sea-island structure is likely to depend on mixing conditions and curing conditions of the epoxy resin and the acrylic rubber. However, when the acrylic rubber is dispersed in the epoxy resin, a sea-island structure is easily formed almost independently of mixing conditions and curing conditions. Therefore, it is possible to increase the degree of freedom of an adhesion operation, curing conditions, and the like.

The content of the acrylic rubber may be 1 mass % or more and 20 mass % or less with respect to the total amount of the main agent. The content of the acrylic rubber is more preferably 5 mass % or more and 15 mass % or less with respect to the total amount of the main agent.

When the acrylic rubber is included, in addition to the adhesive shear strength and the adhesive peel strength, a crosslink density when the adhesive composition is cured is also increased. Thus, it is possible to improve autoclave resistance and chemical resistance of a cured product of the adhesive composition. As a result an adhesive composition that can exhibit sufficient adhesive strength even in a sterilization treatment under high-temperature and high-pressure streams or a sterilization treatment using chemicals can be easily obtained.

Specific examples of the acrylic rubber include AC-3365 (product name; commercially available from Aica Kogyo Company Ltd.).

Here, in Acryset (registered trademark) BPA328 (product name; commercially available from Nippon Shokubai Co., Ltd.) exemplified as a specific example of the epoxy resin, 20±1 (phr) of the acrylic rubber is incorporated with an epoxy equivalent of 230±10 (g/eq.). In Acryset (registered trademark) BPF307 (product name; commercially available from Nippon Shokubai Co., Ltd.) exemplified as a specific example of the epoxy resin, 20±1 (phr) of the acrylic rubber is incorporated with an epoxy equivalent of 210±10 (g/eq.).

As the curing agent, for example, at least one selected from the group consisting of xylylenediamine (also known as xylene diamine), a polyamine, a tertiary amine, and derivatives thereof may be used. The above curing agent including an amine type substance can be referred to as an “amine type curing agent.” Among the amine type curing agents, particularly when xylylenediamine and derivatives thereof are included, a rate of reaction with a main agent increases. Specifically, examples of derivatives of xylylenediamine include an alkylene oxide adduct, a glycidyl ester adduct, a glycidyl ether adduct, a Mannich adduct, an acrylonitrile adduct, an epichlorohydrin adduct, and a xylylenediamine trimer.

As a xylylenediamine used as the curing agent, meta-xylylenediamine having an aromatic framework and being structurally rigid is more preferable.

When xylylenediamine derivatives are used as a curing agent, the content of xylylenediamine derivatives may be 10 mass % or more and 99 mass % or less with respect to the total amount of the curing agent. When xylylenediamine and derivatives thereof are included in such a range, an appropriate reaction rate is obtained, and effects of reducing a reaction with carbon dioxide gas in air and improving adhesive strength can be obtained.

When xylylenediamine derivatives are used, the content of the xylylenediamine derivatives is more preferably 30 mass % or more and 97 mass % or less with respect to the total amount of the curing agent.

As the curing agent used in the adhesive composition of the present embodiment, in addition to the above amine type curing agent, other compounds may be included as the curing agent. Examples of other compounds that can be included in the curing agent include a polyamide resin, imidazoles, and acid anhydrides.

A blending ratio between the main agent and the curing agent is more preferably set such that amounts of an epoxy group in the epoxy resin in the main agent and a functional group of the curing agent which reacts with the epoxy group are equivalent (equivalent blending).

In the epoxy resin, a molecular weight per functional group is referred to as an epoxy equivalent. An amine equivalent of an amine type curing agent is referred to as an active hydrogen equivalent. From the epoxy equivalent and the amine equivalent, a stoichiometric blending ratio in the equivalent blending of the main agent and the curing agent is calculated. The stoichiometric blending ratio is a guideline of an appropriate blending ratio between the main agent and the curing agent. However, a blending ratio between the main agent and the curing agent may be set to be different from the stoichiometric blending ratio, for example, in consideration of the adhesive strength or the like.

In a mass error range of ±50% from the equivalent blending, when the main agent and the curing agent are included at a certain predetermined blending ratio, it is possible to avoid at least one of disadvantages such as oxidative deterioration, hydrolysis, softening deterioration due to heat, curing deterioration, brittle fracture and a reduction in adhesive strength in some cases.

In the adhesive composition of the present embodiment, for example, silica may be included as a filler.

As the silica, for example, spherical silica having an average particle size of 4 μm of or more and 7 μm or less may be used. The content of spherical silica having an average particle size of 4 μm or more and 7 μm or less may be 20 parts by mass or more and 40 parts by mass or less with respect to 100 parts by mass of the main agent. Here, the average particle size is a volume-based average particle size.

The shape of silica can be determined by observation using an electron microscope.

When the adhesive composition includes silica as a filler, since an adhesive layer through which a chemical liquid and water vapor are unlikely to pass is easily formed by silica, the resistance with respect to sterilization becomes higher.

Examples of silicas that can be used for the adhesive composition of the present embodiment include low-viscosity and high-purity spherical silica, EXR-3 (LV) (product name; commercially available from Tatsumori Ltd.), and natural quartz burner fused spherical silica, HPS (registered trademark)-3500 (product name; commercially available from Toagosei Co., Ltd.).

As the inorganic zwitterion exchanger, for example, an inorganic compound including at least one type of metal atom among the group consisting of bismuth (Bi), antimony (Sb), zirconium (Zr), magnesium (Mg), and aluminum (Al) may be used.

Specific examples of such an inorganic zwitterion exchanger include TXE (registered trademark)-600 (product name; commercially available from Toagosei Co., Ltd., Sb- and Bi-based), IXE (registered trademark)-633 (product name; commercially available from Toagosei Co., Ltd., Sb- and Bi-based), IXE (registered trademark)-6107 (product name; commercially available from Toagosei Co., Ltd., Zr- and Bi-based), IXE (registered trademark)-6136 (product name; commercially available from Toagosei Co., Ltd., Zr- and Bi-based), IXEPLAS (registered trademark)-A1 (product name; commercially available from Toagosei Co., Ltd., Zr-, Mg-, and Al-based), IXEPLAS (registered trademark)-A2 (product name; commercially available from Toagosei Co., Ltd., Zr-, Mg-, and Al-based), and IXEPLAS (registered trademark)-B1 (product name; commercially available from Toagosei Co., Ltd., Zr-, and Bi-based).

The content of the inorganic zwitterion exchanger in the adhesive composition of the present embodiment may be 0.1 parts by mass or more and 1.0 part by mass or less with respect to 10 parts by mass of the epoxy resin in the adhesive composition. The content of the inorganic zwitterion exchanger in the adhesive composition of the present embodiment is more preferably 0.2 parts by mass or more and 0.5 parts by mass or less with respect to 10 parts by mass of the epoxy resin.

The adhesive composition of the present embodiment may include 0.1 mass % or more and 5 mass % or less of fumed silica in order to improve thixotropic properties with respect to the total mass of the adhesive composition.

The adhesive composition of the present embodiment may include an additive, for example, a catalyst, an adhesiveness-imparting agent, a solvent, a plasticizer, an antioxidant, a polymerization inhibitor, a surfactant, an antifungal agent, and a coloring agent.

The additives to be added to the adhesive composition of the present embodiment may be added to the main agent in advance or may be added to a mixture of the main agent and the curing agent.

Regarding an example of a method of forming an adhesive layer using the above adhesive composition, an example of adhering and fixing components of an endoscope will be described.

First, a mixture in which a liquid including a main agent and a liquid including a curing agent are mixed at a predetermined ratio is prepared. An inorganic zwitterion exchanger is added to the prepared mixture. Since the inorganic zwitterion exchanger has excellent dispersibility in the main agent compared to, for example, an organic ion exchanger, mixing is easily performed without the viscosity of the mixture increasing significantly. Thus, the mixing workability is improved.

In addition, since the inorganic zwitterion exchanger has excellent dispersibility, it is uniformly dispersed in the main agent.

When the adhesive composition includes a filler or an additive, the inorganic zwitterion exchanger and the filler or the additive may be incorporated into the above mixture.

In this manner, an adhesive composition is obtained.

The obtained adhesive composition is applied to a surface of an adhesion target component of the endoscope on which an adhesive layer is formed. When it is necessary to fix a relative position of the adhesion target component, relative positions of adhesion target components are fixed. Thereafter, the adhesive composition is heated at a predetermined temperature for a predetermined time for curing.

The heating temperature varies depending on the type of main agent and the curing agent included in the adhesive composition, the blending ratio therebetween, and the like. For example, the heating temperature may be 60° C. or higher and 135° C. or lower. When the heating temperature is within the above range, a exiting reaction can occur at a practical rate. In particular, the adhesive composition of the present embodiment includes an amine type curing agent as the curing agent Therefore, in the adhesive composition of the present embodiment, a curing reaction of the main agent rapidly occurs due to the amine type curing agent. The heating time may be 0.5 hours or longer and 3 hours or shorter.

Since the adhesive composition of the present embodiment can be cured at a low temperature as described above, thermal deterioration of components having low heat resistance does not occur.

When heating is completed, the adhesive composition is cured, the adhesive layer is formed, and components of the endoscope are firmly adhered to each other.

A member to be bonded using the above adhesive composition is not particularly limited as long as it is a constituent member of the endoscope. For example, when the adhesive composition of the present embodiment is used, end parts of various tubes inserted into an insertion portion of the endoscope may be fixed to a distal end of the insertion portion or an operation unit. For example, when the adhesive composition of the present embodiment is used, a lens group disposed at a rigid distal end portion of the insertion portion may be fixed to the lens frame or the rigid distal end portion. For example, when the adhesive composition of the present embodiment is used, a fiber bundle inserted into the insertion portion may be fixed to the lens frame or the rigid distal end portion. For example, when the adhesive composition of the present embodiment is used, a CCD and the like incorporated into the rigid distal end portion may be protected and fixed.

According to the same method of forming an adhesive layer, for example, it is possible to seal an imaging device of the endoscope and it is possible to finish and fix the outer surface of the end of a flexible outer tube. In addition, raising and forming an adhesive layer around an observation lens or an illumination lens can be performed by the same method of forming an adhesive layer.

When the outer surface of the constituent member of the endoscope using the adhesive composition of the present embodiment is finished, insertion properties of the constituent member are improved. Specifically, when the end of the flexible outer tube of the insertion portion of the endoscope is tightly bound by a thread from the outside, the end of the flexible outer tube is fixed to a member inside the flexible outer tube. After the adhesive composition is applied to the tightened thread and the adhesive composition is cured, the adhesive layer is formed. Since the adhesive layer covers the thread and is cured, fraying of the thread is prevented. In addition, since a smooth outer surface is formed according to the surface of the adhesive layer, insertion of the insertion portion becomes easier.

The adhesive layer formed in this manner is cured when an epoxy resin including at least one type selected from the group consisting of a bisphenol A type epoxy resin, a bisphenol F type epoxy resin, and a phenol novolak type epoxy resin chemically reacts with an amine type curing agent. Therefore, according to the adhesive layer, favorable adhesive strength and heat resistance are obtained.

In addition, since the adhesive layer is formed by curing the adhesive composition of the present embodiment in which an inorganic zwitterion exchanger is uniformly dispersed, the inorganic zwitterion exchanger is uniformly dispersed also in the adhesive layer.

When a sterilization treatment using a sterilizing gas is performed, the zwitterion exchanger dispersed in the adhesive layer captures anions and cations derived from the sterilizing gas. For example, in the case of a sterilization treatment using hydrogen peroxide plasma, since ions and radical components of the sterilizing gas that come in contact with the adhesive layer are captured (trapped) by the inorganic zwitterion exchanger in the adhesive layer, chemical attack on the adhesive layer is reduced.

As a result, even if a sterilization treatment using a sterilizing gas is repeatedly performed, since the adhesive strength of the adhesive layer does not readily decrease, the adhesive layer has excellent resistance with respect to the sterilization treatment using a sterilizing gas.

In particular, in the adhesive layer obtained by curing the adhesive composition of the present embodiment, compared to a case in which only an inorganic negative (positive) ion exchanger that traps only negative (positive) ions is included, chemical attack is further reduced. Therefore, in the adhesive layer, even if a sterilization treatment using a sterilizing gas is repeated, in particular, the progress of deterioration of the appearance of the adhesive layer is suppressed. As a result, since a user can easily use it repeatedly without anxiety, a practical product lifespan of the constituent member of the endoscope in which the adhesive layer is formed is extended.

Next, an endoscope in which the adhesive composition of the present embodiment is used will be described.

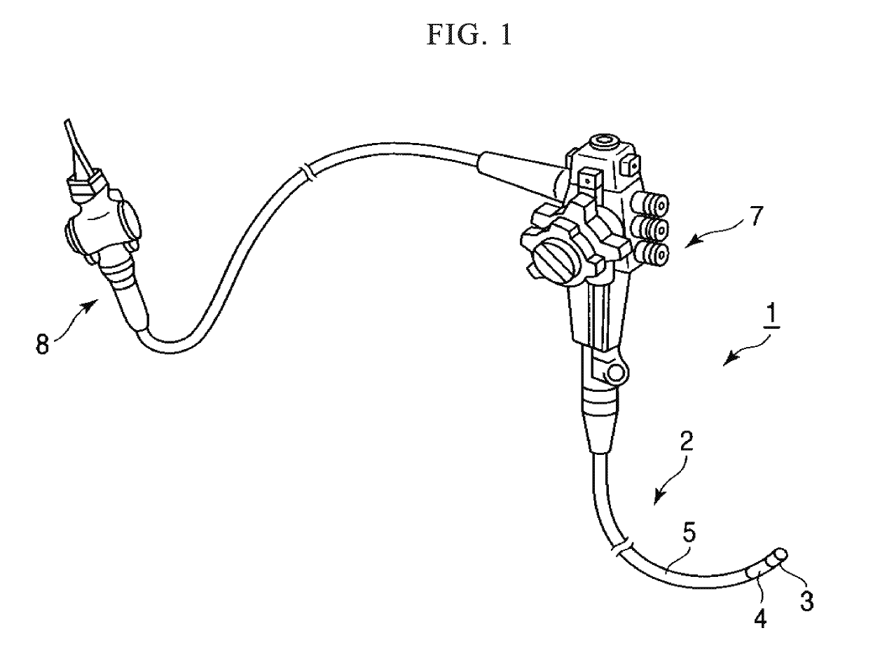

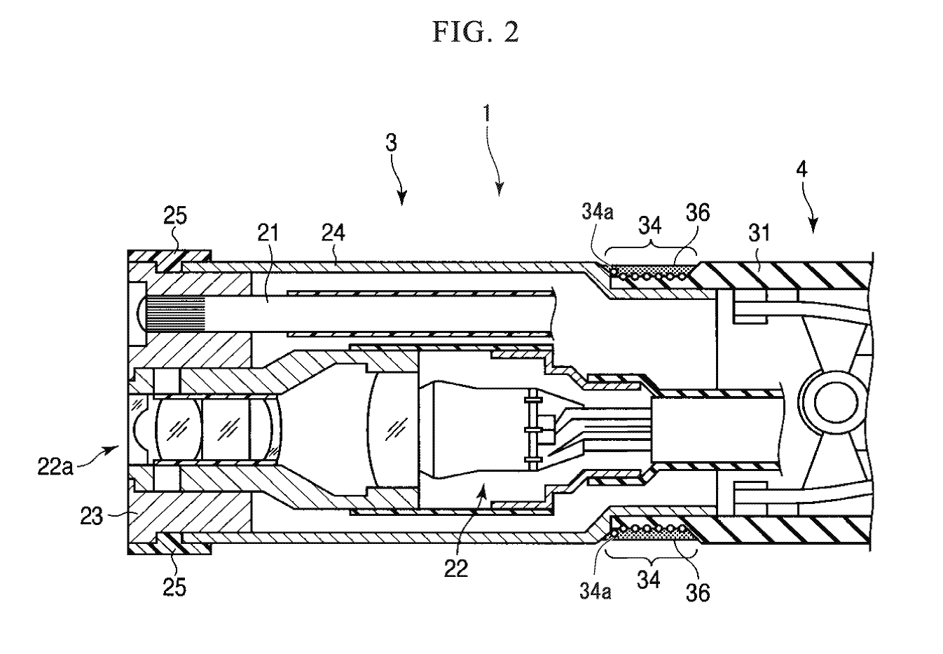

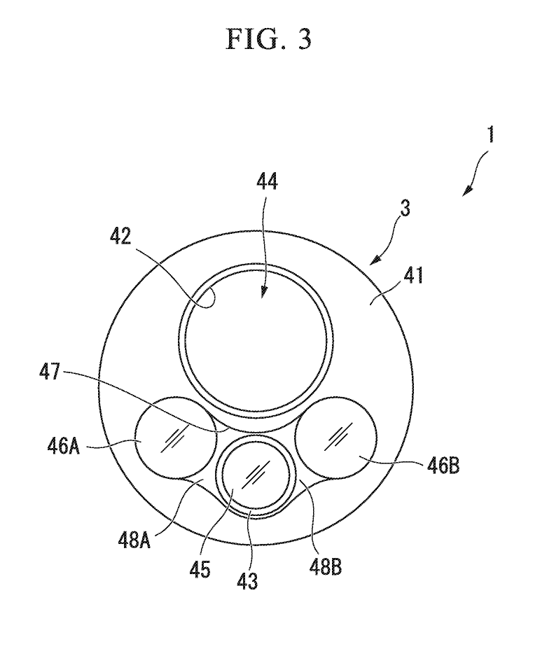

FIG. 1 is a perspective view schematically showing a schematic configuration of an endoscope according to an embodiment of the present invention. FIG. 2 is a cross-sectional view schematically showing an outer tube-fixing part at a distal end portion of the endoscope according to the embodiment of the present invention. FIG. 3 is a front view schematically showing a distal end portion of the endoscope according to the embodiment of the present invention.

Since the drawings are schematic diagrams, the shapes and sizes thereof may be shown in an exaggerated manner.

As shown in FIG. 1, an endoscope 1 of the present embodiment includes an insertion portion 2, an operation unit 7, and a universal cord 8. The insertion portion 2 is formed in an elongated shape. The insertion portion 2 is inserted into a subject's body. The operation unit 7 is connected to the insertion portion 2. The universal cord 8 is electrically connected to the operation unit 7. The universal cord 8 supplies illumination light.

The insertion portion 2 includes a distal end portion 3, a bending portion 4, and a flexible tube portion 5 in that order from the distal end side in die insertion direction toward the operation unit 7 on the proximal end side.

The distal end portion 3 disposed at the distal end of the insertion portion 2 emits illumination light from the distal end thereof and receives reflected light from the inside of the body.

An optical fiber that transmits light received at the distal end portion 3 is accommodated in the flexible tube portion 5 and the bending portion 4.

The bending portion 4 is bent according to an operation input from the operation unit 7.

In such an endoscope 1, a member to be bonded using the adhesive composition of the present embodiment is not particularly limited as long as the member is a constituent member of the endoscope 1. A manner of use in the present embodiment will be exemplified below.

As shown in FIG. 2, at the distal end portion 3 of the endoscope 1, a light guide fiber 21 configured to supply illumination light and a cylindrical block-like rigid distal end portion 23 that holds an imaging unit 22 are provided. A distal end cover 24 is fitted to a side surface of the rigid distal end portion 23. At a fitting part of the rigid distal end portion 23 and the distal end cover 24, an adhesive layer 25 in which the above adhesive composition are cured is provided. The adhesive layer 25 bonds the rigid distal end portion 23 and the distal end cover 24 to each other.

On the proximal end side of the distal end cover 24, a bending rubber 31 as a cylindrical outer tube covering the outer circumference of the bending portion 4 is externally inserted. At the part into which the bending rubber 31 is externally inserted, a thread 34a is wound from above the bending rubber 31 and a thread-winding portion 34 is formed. The bending rubber 31 is tightly bound by the thread 34a of the thread-winding portion 34. The thread-winding portion 34 fixes the bending rubber 31 to the distal end cover 24.

On the outer circumference of the thread-winding portion 34, an adhesive layer 36 obtained by curing the above adhesive composition is formed. The adhesive layer 36 prevents fraying of the thread 34a in the thread-winding portion 34.

In addition, the adhesive layer 36 covers the thread-winding portion 34 along side surfaces of the distal end cover 24 and the bending rubber 31. The adhesive layer 36 covers the thread-winding portion 34 and forms a smooth outer surface. Accordingly, at the adhesive layer 36, during insertion of the insertion portion 2, the distal end portion 3 and the bending portion 4 come in contact with a living body and can slide smoothly.

Although not shown, in the endoscope 1, using the above adhesive composition, end parts of various tubes inserted into the insertion portion 2 of the endoscope 1 may be fixed to the distal end of the insertion portion 2 or the operation unit 7.

In the endoscope 1, using the above adhesive composition, a lens group 22a (refer to FIG. 2) and the like disposed at the rigid distal end portion 23 of the insertion portion 2 may be fixed to the lens frame or the rigid distal end portion 23.

In the endoscope 1, using the above adhesive composition, a fiber bundle inserted into the insertion portion 2 may be fixed to the lens frame or the rigid distal end portion 23.

In the endoscope 1, using the above adhesive composition, a charge-coupled device (CCD) and the like in the imaging unit 22 incorporated into the distal end portion 3 may be protected, fixed, and sealed.

Although not shown, in the endoscope 1, the outer circumference of the part connecting the bending portion 4 to the flexible tube portion 5 has the same configuration as the outer circumference of the portion connecting the distal end portion 3 to the bending portion 4. Specifically, a thread-winding portion is formed in the part connecting the bending portion 4 to the flexible tube portion 5. The same adhesive composition as above is applied to the outer circumference of the thread-winding portion. When the adhesive composition is cured, the same adhesive layer as above is formed. According to the adhesive layer also, similarly to the above, fraying of the thread of the thread-winding portion is prevented and a smooth outer surface for improving insertion properties is formed.

In the endoscope 1, using the above adhesive composition, an imaging element of the endoscope may be sealed.

In the endoscope 1, by raising the adhesive composition around an observation lens or an illumination lens, corners of the outer circumference of the lens may be smoothed.

The adhesive composition of the present embodiment may be disposed around the lens frame in the distal end portion 3 of the endoscope 1.

As shown in FIG. 3, an insulating member 41 is disposed at the distal end of the distal end portion 3 of the endoscope 1. A first opening 44 and a second opening 47 penetrate through the insulating member 41. The first opening 44 communicates with a forceps channel 42. Inside the second opening 47, an objective lens frame 43, and illumination lenses 46A and 46B are disposed.

The objective lens frame 43 holds an objective lens 45. The objective lens frame 43 is disposed at the center part of the second opening 47.

The illumination lenses 46A and 46B are disposed at both ends of the second opening 47.

In the second opening 47, both the objective lens frame 43, and the illumination lenses 46A and 46B are adhered to the inner circumferential surface of the second opening 47 using the adhesive composition of the present embodiment.

In addition, inside the second opening 47, in a space between the objective lens frame 43 and the illumination lens 46A, and a space between the objective lens frame 43 and the illumination lens 46B, adhesive layers 48A and 48B obtained by filling in and solidifying the adhesive composition of the present embodiment are formed.

The adhesive layer 48A adheres and fixes the objective lens frame 43 and the illumination lens 46A to each other. The adhesive layer 48B adheres and fixes the objective lens frame 43 and the illumination lens 46B to each other. The adhesive layer 48A seals a space between the objective lens frame 43 and the illumination lens 46A. The adhesive layer 48B seals a space between the objective lens frame 43 and the illumination lens 46B.

In this manner, in the endoscope 1, the adhesive layer of the adhesive composition of the present embodiment is used for various applications. The adhesive layer in the present embodiment may be used for applications, for example, bonding between constituent members, fixing the outer tube and the thread, finishing the outer surface at the end of the outer tube, sealing the imaging element, and a smoothening treatment of corners of the outer circumference of the lens.

Since the adhesive layer in the present embodiment is formed by curing the adhesive composition of the present embodiment, the adhesive layer in the present embodiment has an excellent sterilization resistance even after the sterilization treatment using, for example, hydrogen peroxide plasma. The adhesive layer in the present embodiment can maintain a favorable adhesive strength and appearance even after the sterilization treatment.

In addition, since the adhesive composition of the present embodiment includes the inorganic zwitterion exchanger, the adhesive composition of the present embodiment has a viscosity at which, for example, a coating operation in an application for bonding constituent members of the endoscope 1 to each other and a coating operation in an application for finishing the outer surface are easily performed.

In addition, since the adhesive composition of the present embodiment includes the inorganic zwitterion exchanger, even if a substance causing chemical attack is an anion or a cation, the adhesive composition of the present embodiment can trap a substance causing chemical attack. Therefore, even if the type of sterilizing gas is changed and thus the type of ions generated during sterilization is changed, the adhesive composition of the present embodiment has a favorable sterilization resistance. As a result, the endoscope in which the adhesive composition of the present embodiment is used exhibits a high sterilization resistance with respect to a sterilization treatment using various sterilizing gases.

### Second Embodiment

Next, an adhesive composition, an ultrasonic transducer, and an ultrasonic endoscope according to a second embodiment of the present invention will be described.

As one type of the endoscope, an ultrasonic endoscope is known. The ultrasonic endoscope has an ultrasonic transducer in which an acoustic matching layer is formed in order for a submucosal interior to be observed.

The acoustic matching layer in the ultrasonic transducer needs to have appropriate acoustic characteristics according to acoustic characteristics of an observation target. As a base material of the acoustic matching layer, an epoxy resin, a urethane resin, and the like are used in many cases.

For example, in Japanese Unexamined Patent Application, First Publication No. 2014-188009, an ultrasound probe used for an ultrasound image diagnostic layer is described. The ultrasound probe has an acoustic matching layer formed by adding zinc oxide, titanium oxide, silica, alumina, red iron oxide, ferrite, tungsten oxide, yttrium oxide, barium sulfate, tungsten, molybdenum, or the like to an epoxy resin, and performing kneading to achieve uniformity. The acoustic matching layer is adhered to a piezoelectric element or another acoustic matching layer by an epoxy type adhesive.

Like other medical endoscopes, since the ultrasonic endoscope is inserted into the body and then used, the ultrasonic endoscope undergoes, for example, a sterilization treatment such as hydrogen peroxide plasma sterilization at low temperatures. Therefore, there is a risk of the acoustic matching layer and the adhesive layer used in the ultrasonic endoscope deteriorating due to chemical attack during sterilization. For example, when the acoustic matching layer deteriorates, since acoustic characteristics of the acoustic matching layer are changed, it may not be possible to acquire an accurate ultrasound image. When the adhesive layer deteriorates, there is a risk of an adhesive counterpart member coming off.

Therefore, in the ultrasonic endoscope, it is strongly required to improve the resistance with respect to a sterilization treatment using a sterilizing gas. Improvement in the resistance of the medical instrument leads to reduction in medical costs according to improvement in cost performance of the medical instrument.

The adhesive composition of the present embodiment is suitably used in the ultrasonic endoscope having the above problems.

The adhesive composition of the present embodiment further includes an inorganic filler in addition to the adhesive composition of the first embodiment. That is, the adhesive composition of the present embodiment includes the same epoxy resin and inorganic zwitterion exchanger as in the first embodiment and an inorganic filler.

Differences from the first embodiment will be mainly described below.

Hereinafter, for simplicity, the adhesive composition of the first embodiment will be referred to as an “adhesive composition (I),” and the adhesive composition of the present embodiment will be referred to as an “adhesive composition (II)” in some cases.

As the inorganic filler in the present embodiment, an appropriate inorganic material that can be included in the adhesive composition (I) of the first embodiment is used. The inorganic filler may be an insulator or a conductor.

As the inorganic filler, a material having a higher specific gravity than a cured product of the epoxy resin included in the adhesive composition (I) may be used. In this case, when the inorganic filler is included, it is possible to increase the specific gravity of the cured product of the adhesive composition (II). When the content of the inorganic filler in the adhesive composition (II) is changed, it is possible to change the specific gravity of the cured product of the adhesive composition (II).

The specific gravity of the cured product of the adhesive composition (II) corresponds to, for example, an acoustic impedance, which is one of the acoustic characteristics of the cured product of the adhesive composition (II). When the specific gravity of the inorganic filler is higher, a smaller amount of the inorganic filler is included, and thus an acoustic impedance required for the cured product of the adhesive composition (II) is obtained. When the specific gravity of the inorganic filler is higher and thus the content of the inorganic filler is reduced, coating performance and formability when the adhesive composition (II) is formed are improved.

For example, the specific gravity of the inorganic filler in the adhesive composition (II) may be 3 or more.

Specific examples of a suitable inorganic filler of the adhesive composition (II) include at least one type of inorganic filler selected from the group consisting of alumina, zirconia, silicon nitride, silicon carbide, tungsten trioxide, diamond, sapphire, aluminum nitride, boron nitride, and magnesium oxide.

Examples of alumina that can be used in the adhesive composition (II) include ionic impurity reduction high-sphericity alumina, such as Denka spherical alumina DAW-07, DAW-05 (product name; commercially available from Denka Company Ltd.).

Examples of zirconia that can be used in the adhesive composition (II) include zirconia beads DZB ϕ7 (product name; commercially available from Daiken Chemical Co., Ltd.), and micro zirconia beads NZ10 (product name; commercially available from Niimi Sangyo CO., Ltd.).

The inorganic filler may be spherical particles having an aspect ratio of 0 or more and less than 0.5. In this case, the fluidity of the adhesive composition (II) becomes favorable and the formability is improved. When the fluidity and formability become favorable, the shape of a mold is accurately transferred when the adhesive composition (II) is formed and cured. When an accurate mold shape is obtained, for example, stable acoustic performance is obtained.

When the aspect ratio of the inorganic filler is 0.5 or more, the viscosity of the adhesive composition becomes too low due to an interaction between inorganic filler particles in the adhesive composition or between an inorganic filler and other particles. Therefore, there is a risk of the formability of the adhesive composition being reduced.

In the adhesive composition (II), 30 parts by mass or more and 300 parts by mass or less of the inorganic filler may be included with respect to 10 parts by mass of the epoxy resin. In this case, according to the content of the inorganic filler, it is easy to optimize acoustic characteristics of the cured product of die adhesive composition (II). In addition, since the fluidity of the adhesive composition (II) becomes favorable, the formability of the adhesive composition (II) is improved.

When the content of the inorganic filler is less than 30 parts by mass, there is a risk of an acoustic impedance required for the acoustic matching layer of the ultrasonic transducer for medical applications being not easily obtained.

When the content of the inorganic filler exceeds 300 parts by mass, since the viscosity of the adhesive composition becomes too low, there is a risk of the formability of the adhesive composition being reduced.

In the adhesive composition (II), with respect to 10 parts by mass of the epoxy resin, 0.5 parts by mass or more and 5 parts by mass or less of the inorganic zwitterion exchanger may be included. In this case, when the inorganic filler is included, even if a relative content in the cured product is reduced, a favorable resistance with respect to a sterilizing gas is maintained and favorable formability is obtained.

When the content of the inorganic zwitterion exchanger is less than 0.5 parts by mass, since an ability of trapping hydrogen peroxide gas in the adhesive composition is lowered, there is a risk of the durability of the cured product of the adhesive composition being further reduced.

When the content of the inorganic zwitterion exchanger exceeds 5 parts by mass, since the viscosity of the adhesive composition becomes too low due to an interaction with the inorganic filler in the adhesive composition, there is a risk of the formability of the adhesive composition being reduced.

Like the cured resin layer using the adhesive composition (I) according to the first embodiment, the cured resin layer formed by curing the adhesive composition (II) of each of the above configurations is cured when an epoxy resin including at least one type selected from the group consisting of a bisphenol A type epoxy resin, a bisphenol F type epoxy resin, and a phenol novolak type epoxy resin chemically reacts with an amine type curing agent. Therefore, the adhesive composition (II) has favorable adhesive strength and heat resistance.

In addition, in the adhesive composition (II) of each of the above configurations, since the inorganic zwitterion exchanger is uniformly dispersed, even in the cured resin layer obtained by curing the adhesive composition (II), the inorganic zwitterion exchanger is uniformly dispersed. Therefore, even if a sterilization treatment using a sterilizing gas is repeatedly performed as in the cured resin layer obtained by curing the adhesive composition (I), the cured resin layer of the adhesive composition (II) has an adhesive strength that is unlikely to decrease and has excellent resistance.

Next, an ultrasonic transducer and an ultrasonic endoscope according to the present embodiment in which the adhesive composition (II) is used will be described.

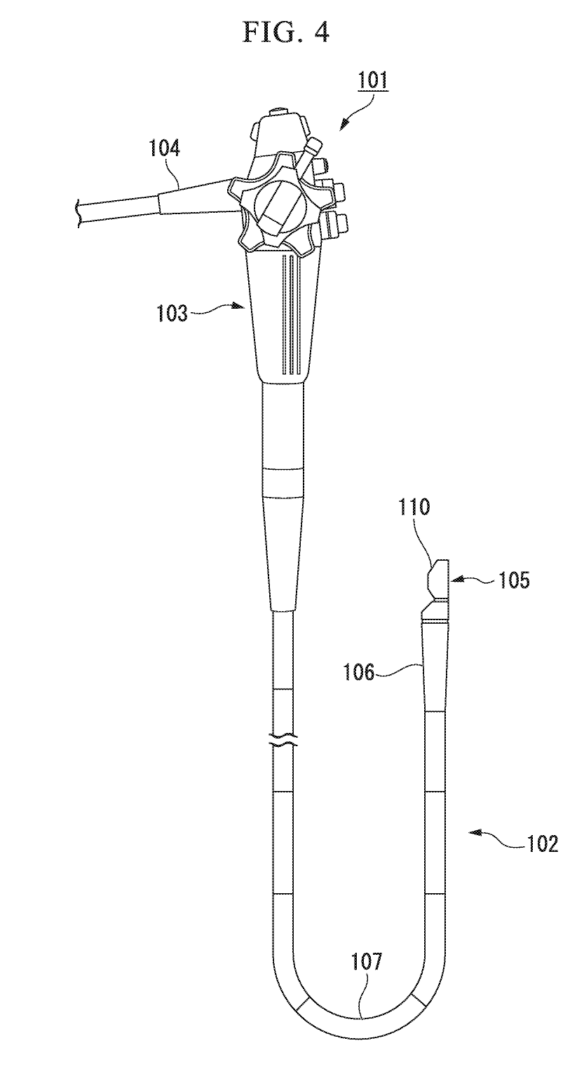

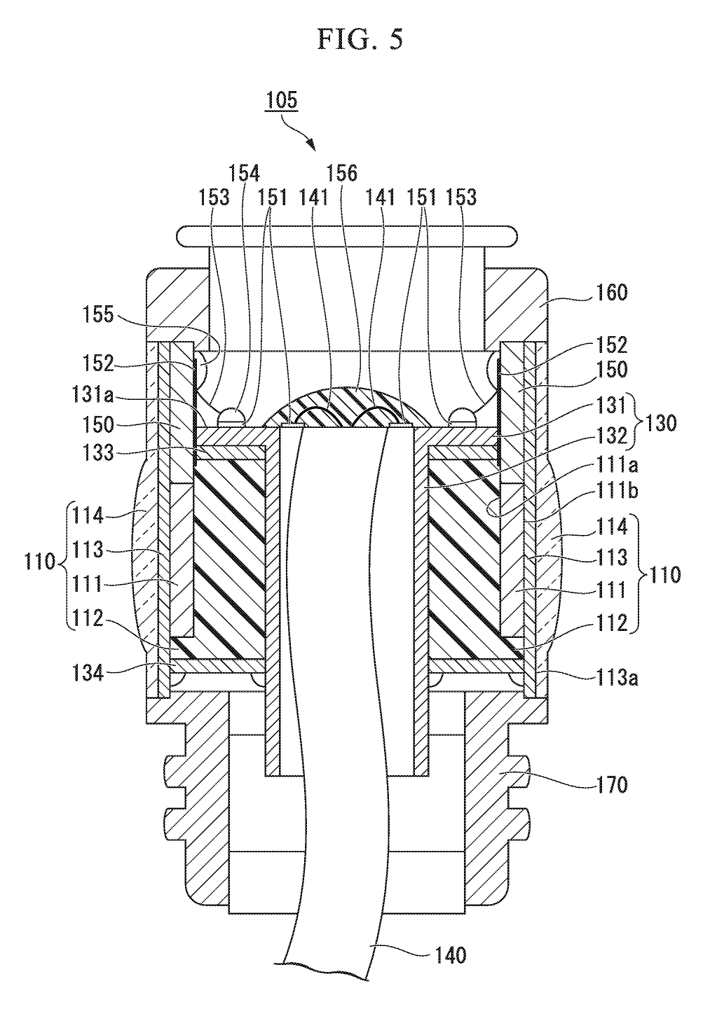

FIG. 4 is a front view schematically showing a schematic configuration of an ultrasonic endoscope according to the second embodiment of the present invention. FIG. 5 is a cross-sectional view schematically showing a configuration of main parts of the ultrasonic endoscope according to the second embodiment of the present invention.

As shown in FIG. 4, an ultrasonic endoscope 101 of the present embodiment includes an insertion portion 102, an operation unit 103, and a universal cord 104. The insertion portion 102 is formed in an elongated shape. The insertion portion 102 is inserted into a subject's body. The operation unit 103 is connected to a proximal end of the insertion portion 102. The universal cord 104 extends from the operation unit 103.

The insertion portion 102 has a configuration in which a rigid distal end portion 105, a bendable bending portion 106, and a flexible tube portion 107 that has a small diameter, is long and has flexibility are connected in that order from the distal end of the insertion portion 102.

As shown in FIG. 5, the rigid distal end portion 105 includes a cylindrical member 130, and a plurality of ultrasonic transducers 110.

The cylindrical member 130 includes an annular flange 131 and a cylindrical portion 132 that extends from the center edge of the flange 131 in a direction (direction from the top to the bottom in the figure) of the flexible tube portion 107 (not shown).

A coaxial cable 140 is inserted into the cylindrical portion 132 of the cylindrical member 130.

The ultrasonic transducer 110 is a device part that emits ultrasonic waves to a subject A plurality of ultrasonic transducers 110 are arranged in the circumferential direction along the outer circumferential surface of the cylindrical member 130.

The ultrasonic transducers 110 each include a piezoelectric element 111, a backing material 112, an acoustic matching layer 113 (cured resin layer), an acoustic lens 114, and an electrode (not shown).

The piezoelectric element 111 generates ultrasonic vibration when a voltage is applied by an electrode (not shown). The piezoelectric element 111 in the present embodiment is formed in a flat plate shape. One plate surface 111a of the piezoelectric element 111 is disposed at a position that faces the cylindrical portion 132 in the radial direction of the cylindrical member 130.

The backing material 112 is a member for absorbing vibration from the plate surface 111a directed inward in the radial direction among ultrasonic vibrations generated by the piezoelectric element 111. The backing material 112 is filled between the cylindrical portion 132 and the piezoelectric element 111.

As a material of the backing material 112, a resin material having appropriate vibration absorption characteristics is used. The resin material used for the backing material 112 is more preferably, for example, a material having resistance with respect to a sterilization treatment using a sterilizing gas as in the adhesive composition (I).

In the axial direction, the backing material 112 is inserted between annular members 133 and 134 into which the cylindrical portion 132 is inserted.

The annular member 133 is adjacent to the flange 131, and is attached so that it is in contact with a substrate 150 that extends from the piezoelectric element 111 in the distal end direction of the rigid distal end portion 105.

The annular member 134 is attached so that it is in contact with the acoustic matching layer 113 to be described below at a position closer to the flexible tube portion 107 (not shown) than the piezoelectric element 111.

The acoustic matching layer 113 is a layered part that reduces a difference between acoustic impedances of a subject and the piezoelectric element 111. When the acoustic impedance of the acoustic matching layer 113 is appropriately set according to the acoustic impedance of the subject, reflection of ultrasonic waves at the subject is reduced.

The acoustic matching layer 113 is provided to cover at least a plate surface 111b on the side opposite to the plate surface 111a in the piezoelectric element 111. Therefore, ultrasonic waves emitted from the plate surface 111b outwardly in the radial direction are efficiently introduced into the subject through the acoustic matching layer 113.

The acoustic matching layer 113 may be composed of a single layer or a plurality of layers.

The acoustic matching layer 113 includes a layer formed of the adhesive composition (II). The acoustic matching layer 113 may include a layer formed of the adhesive composition (I).

The acoustic matching layer 113 is formed by curing a resin composition such as the adhesive composition (II) that is appropriately laminated using, for example, an appropriate mold.

In the acoustic matching layer 113, when the cured resin layer obtained by curing the adhesive composition (II) is used as the acoustic matching layer 113, the resistance with respect to a sterilization treatment using a sterilizing gas is improved. Therefore, even if a sterilization treatment using a sterilizing gas is repeatedly performed on the ultrasonic transducer 110 and the ultrasonic endoscope 101, acoustic characteristics of the acoustic matching layer 113 are changed, and it is possible to prevent an accurate ultrasound image from being unable to be acquired. Therefore, the durability of the ultrasonic transducer 110 and the ultrasonic endoscope 101 is improved.

The acoustic lens 114 converges ultrasonic waves that are generated in the piezoelectric element 111 and propagate outwardly in the radial direction through the acoustic matching layer 113 and emits the ultrasonic waves to the outside. The acoustic lens 114 is formed in a shape appropriate for converging ultrasonic waves. The acoustic lens 114 is provided to cover the acoustic matching layer 113 from the outside in the radial direction.

In the flange 131 of the cylindrical member 130, on a surface 131a in a direction opposite to the annular member 133, a plurality of electrode pads 151 are provided.

A wiring 141 that extends from the coaxial cable 140 is linked to the electrode pad 151. The electrode pad 151 and an electrode layer 152 provided on the substrate 150 are linked by a wire 153. The electrode pad 151 and the wire 153 are bonded by a solder 154. The electrode layer 152 and the wire 153 are bonded by a solder 155.

The entire linking part between the electrode pad 151 and the wiring 141 is covered with a potting resin 156 in order to prevent the wiring 141 from coming off from the electrode pad 151, for example, when a load is applied to the coaxial cable 140.

At the distal end of the rigid distal end portion 105, a distal end structure member 160 is provided to block the linking part between the electrode pad 151 and the wiring 141. In addition, the rigid distal end portion 105 is connected to the bending portion 106 (not shown in FIG. 5) via a connecting member 170.

For example, the ultrasonic transducer 110 having such a configuration is produced as follows.

The piezoelectric element 111 in which electrodes (not shown) are provided on the plate surfaces 111a and 111b and the acoustic matching layer 113 that is formed in advance are bonded. Thereafter, the substrate 150 is attached to the piezoelectric element 111 so that it extends in the planar direction. Further, the annular members 133 and 134 are disposed at predetermined positions.

Thereafter, a resin composition for forming the backing material 112 is poured between the piezoelectric element 111 surrounded by the annular members 133 and 134 and the cylindrical member 130. As the resin composition, for example, the adhesive composition (I) may be used.

When a curing treatment for curing a resin composition is performed, the backing material 112 is formed.

Then, on a surface 113a in a direction opposite to the piezoelectric element 111 in the acoustic matching layer 113, the acoustic lens 114 is formed. In this manner, the ultrasonic transducer 110 is produced.

The ultrasonic transducer 110 of the present embodiment has a cured resin layer obtained by curing the adhesive composition (II) of the present embodiment as the acoustic matching layer 113. Therefore, the resistance with respect to a sterilization treatment using a sterilizing gas on the ultrasonic transducer 110 and the ultrasonic endoscope 101 is improved. Specifically, even if a sterilization treatment is repeatedly performed, disturbance is unlikely to occur in an image obtained by the ultrasonic endoscope 101 during examination and diagnosis.

In addition, the adhesive composition (II) of the present embodiment has appropriate acoustic characteristics and improved fluidity by setting, for example, at least one type of inorganic filler, the aspect ratio of the inorganic filler, the content of the inorganic filler with respect to the epoxy resin, and the content of the inorganic zwitterion exchanger with respect to the epoxy resin to be within an appropriate range as described above. Therefore, according to the adhesive composition (II), formability when the adhesive composition (II) for use in the ultrasonic transducer 110 is formed is further improved.

As described above, when the adhesive composition (II) is used, it is possible to provide the ultrasonic transducer 110 and the ultrasonic endoscope 101 through which it is possible to achieve both improvement in the resistance with respect to a sterilization treatment using a sterilizing gas and stability of acoustic characteristics in the acoustic matching layer 113.

### Third Embodiment

Next, an ultrasonic transducer according to a third embodiment of the present invention will be described.

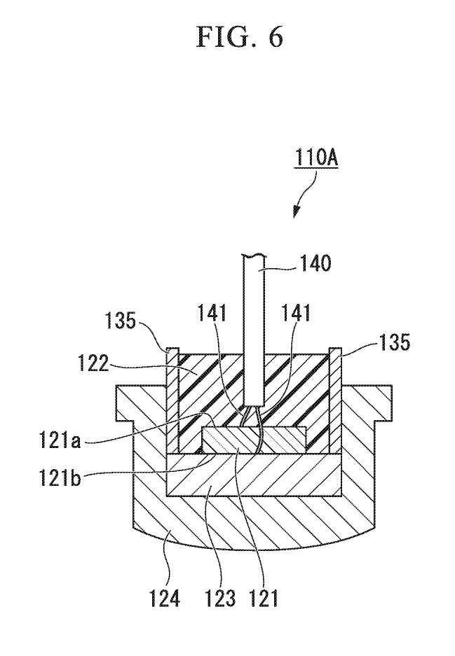

FIG. 6 is a cross-sectional view schematically showing a schematic configuration of the ultrasonic transducer according to the third embodiment of the present invention.

As shown in FIG. 6, an ultrasonic transducer 110A of the present embodiment includes a piezoelectric element 121, a backing material 122, an acoustic matching layer 123 (cured resin layer), and an acoustic lens 124 in place of the piezoelectric element 111, the backing material 112, the acoustic matching layer 113, and the acoustic lens 114 of the ultrasonic transducer 110 of the second embodiment.

Differences from the second embodiment will be mainly described below.

The piezoelectric element 121 has a disk shape. On surfaces 121a and 121b on both sides of the piezoelectric element 121, an electrode (not shown) for applying a voltage to the piezoelectric element 121 is provided. The wiring 141 that extends from the coaxial cable 140 is linked to the electrode (not shown).

While the distal end of the coaxial cable 140 and the wirings 141 are included therein, the backing material 122 is provided to cover one surface 121a of the piezoelectric element 121 and side surfaces of the piezoelectric element 121.

As a material of the backing material 122, the same material as the backing material 112 of the second embodiment can be used.

The acoustic matching layer 123 is a disk having a larger diameter than the piezoelectric element 121. The acoustic matching layer 123 is provided in contact with the other surface 121b of the piezoelectric element 121. On the outer circumferential part of the surface of the acoustic matching layer 123 in contact with the piezoelectric element 121, a cylindrical member 135 having the same diameter as the outer diameter of the acoustic matching layer 123 stands up. The inner circumferential surface of the cylindrical member 135 is in close contact with the side surface of the backing material 122.

As a material of the acoustic matching layer 123, the same material as that of the acoustic matching layer 123 of the second embodiment can be used.

The acoustic lens 124 has a circular lens area in a plan view according to the piezoelectric element 121 and the acoustic matching layer 123 having a disk shape. However, the acoustic lens 124 is formed in a cap shape that covers side surfaces of the acoustic matching layer 123 and some of side surfaces of the cylindrical member 135.

In order to produce the ultrasonic transducer 110A of the present embodiment, first, the acoustic matching layer 123 is bonded to the surface 121b of the piezoelectric element 121. Thereafter, a resin composition for forming the backing material 122 is poured into a space surrounded by the acoustic matching layer 123 and the cylindrical member 135 that stands on the outer circumferential part of the acoustic matching layer 123. When the resin composition is cured, the backing material 122 is formed.

Thereafter, the acoustic lens 124 is formed to cover outer surfaces of the acoustic matching layer 123, and the cylindrical member 135. In this manner, the ultrasonic transducer 110A is produced.

The ultrasonic transducer 110A of the present embodiment can be used in the ultrasonic endoscope 101 of the second embodiment in place of the ultrasonic transducer 110 of the second embodiment.

The ultrasonic transducer 110A of the present embodiment has the same effects as that of the second embodiment because only its outer shape is different from that of the ultrasonic transducer 110A of the second embodiment.

Here, while a case in which the adhesive compositions of the first and second embodiments are used for the endoscope and the ultrasonic endoscope has been exemplified in the description of the above embodiments, the adhesive compositions of the first and second embodiments may be used for various medical instruments on which a sterilization treatment using a sterilizing gas is performed or devices other than medical instruments.

In particular, the adhesive composition of the second embodiment may be used for ultrasonic transducers for various applications on which a sterilization treatment using a sterilizing gas is performed.

### EXAMPLES

### Examples Related to the First Embodiment

Examples 1 to 7 of the adhesive composition according to the first embodiment will be described below together with Comparative Examples 1 to 5.

In the following [Table 1], compositions and evaluation results of the adhesive compositions of Examples 1 to 7, and Comparative Examples 1 to 5 are shown.

### [Table 1]

The only difference between the compositions of Examples 1 to 7, and Comparative Examples 1 to 5 was the type of ion exchanger included in the adhesive composition.

In Examples 1 to 7, and Comparative Examples 1 to 5, aside from the ion exchanger, types of a main agent, a curing agent, and a filler were the same.

In Examples 1 to 7, and Comparative Examples 1 to 5, the composition of the adhesive composition included 103 parts by mass of the main agent, 40 parts by mass of the curing agent, 40 parts by mass of the filler, and 5 parts by mass of the ion exchanger.

In the following [Table 2], configurations the same as in the examples and the comparative examples are shown.

[Table 2]

As shown in [Table 2], the main agent was formed by mixing 10 parts by mass of Adeka resin EP (registered trademark)-4100E (product name; commercially available from ADEKA), 3 parts by mass of Acryset (registered trademark) BPF307 (product name; commercially available from Nippon Shokubai Co., Ltd.), 60 parts by mass of Acryset (registered trademark) BPA328 (product name; commercially available from Nippon Shokubai Co., Ltd.), and 30 parts by mass of jER (registered trademark) 152 (product name; commercially available from Mitsubishi Chemical Corporation).

The bisphenol A type epoxy resin was included in the above Adeka resin EP (registered trademark)-4100E (product name; commercially available from ADEKA) and Acryset (registered trademark) BPA328 (product name; commercially available from Nippon Shokubai Co., Ltd.).

The bisphenol F type epoxy resin was included in the above Acryset (registered trademark) BPF307 (product name; commercially available from Nippon Shokubai Co., Ltd.).

The bisphenol novolak type epoxy resin was included in the above jER (registered trademark) 152 (product name; commercially available from Mitsubishi Chemical Corporation).

The acrylic rubber was included in the above Acryset (registered trademark) BPA328 (product name; commercially available from Nippon Shokubai Co., Ltd.) and Acryset (registered trademark) BPF307 (product name; commercially available from Nippon Shokubai Co., Ltd.).

As the curing agent, 40 parts by mass of a mixture of meta-xylylenediamine and meta-xylylenediamine derivatives (commercially available from Mitsubishi Gas Chemical Company, Inc.) was used.

As the filler, 40 parts by mass of EXR-3(LV) (product name; commercially available from Tatsumori Ltd.) as low-viscosity and high-purity spherical silica was used.

Specific types of the ion exchangers of the examples and the comparative examples are shown in the following [Table 3].

### [Table 3]

### Examples 1 to 7

As shown in [Table 1], as the ion exchangers of Examples 1 to 7, inorganic zwitterion exchangers A, B, C, D, E, F, and G were used.

As shown in [Table 3], as the inorganic zwitterion exchanger A of Example 1, IXE (registered trademark)-600 (product name; commercially available from Toagosei Co., Ltd.) was used.

As the inorganic zwitterion exchanger B of Example 2, IXE (registered trademark)-633 (product name; commercially available from Toagosei Co., Ltd.) was used.

Both the inorganic zwitterion exchangers A and B were a Sb- and Bi-based inorganic compound.

As the inorganic zwitterion exchanger C of Example 3, IXE (registered trademark)-6107 (product name; commercially available from Toagosei Co., Ltd.) was used.

As the inorganic zwitterion exchanger D of Example 4, IXE (registered trademark)-6136 (product name; commercially available from Toagosei Co., Ltd.) was used.

Both the inorganic zwitterion exchangers C and D were a Zr- and Bi-based inorganic compound.

As the inorganic zwitterion exchanger E of Example 5, IXEPLAS (registered trademark)-A1 (product name; commercially available from Toagosei Co., Ltd.) was used.

As the inorganic zwitterion exchanger F of Example 6, IXEPLAS (registered trademark)-A2 (product name; commercially available from Toagosei Co., Ltd.) was used.

Both the inorganic zwitterion exchangers E and F were a Zr-, Mg-, and Al-based inorganic compound.

As the inorganic zwitterion exchanger G of Example 7, IXEPLAS (registered trademark)-B1 (product name; commercially available from Toagosei Co., Ltd.) was used.

The inorganic zwitterion exchanger G was a Zr- and Bi-based inorganic compound.

### Comparative Examples 1 to 5

As shown in [Table 1], as ion exchangers of Comparative Examples 1 to 5, an inorganic cation exchanger a, an inorganic anion exchanger b, an organic zwitterion exchanger c, an organic cation exchanger d, and an organic anion exchanger e, which were different from the inorganic zwitterion exchangers, were used, respectively.

As shown in [Table 3], as the inorganic cation exchanger a of Comparative Example 1, IXE (registered trademark)-100 (product name; commercially available from Toagosei Co., Ltd.) was used.

As the inorganic anion exchanger b of Comparative Example 2, EXE (registered trademark)-800 (product name; commercially available from Toagosei Co., Ltd.) was used.

Both the inorganic cation exchanger a and the inorganic anion exchanger b were a Zr-based inorganic compound.

As the organic zwitterion exchanger c of Comparative Example 3, Diaion (registered trademark) AMP03 (product name; commercially available from Mitsubishi Chemical Corporation) was used.

As the organic cation exchanger d of Comparative Example 4, Diaion (registered trademark) PK208 (product name; commercially available from Mitsubishi Chemical Corporation) was used.

As the organic anion exchanger e of Comparative Example 5, Diaion (registered trademark) PA306S (product name; commercially available from Mitsubishi Chemical Corporation) was used.

All of the organic zwitterion exchanger c, the organic cation exchanger d, and the organic anion exchanger e were a crosslinked polystyrene.

The above main agents, curing agents, fillers, and ion exchangers were mixed at the above mass ratios, and thereby the adhesive compositions of Examples 1 to 7 and Comparative Examples 1 to 5 were obtained.

### [Evaluation]

The adhesive composition of the examples and the comparative examples was applied to the thread-winding portion 34 of the above endoscope 1.

The applied adhesive composition was heated and cured. When the adhesive composition was cured, the adhesive layer was formed.

Accordingly, as test samples, insertion portions of an endoscope having an adhesive layer covering the thread-winding portion 34 were obtained.

The test samples were subjected to 300 sterilization treatments (300 times), using Sterrad (registered trademark) NX (registered trademark) (product name; commercially available from Johnson & Johnson), which is a sterilizer used to perform hydrogen peroxide plasma sterilization. Sterilization conditions were set to be in an advanced mode each time.

After 300 sterilization treatments were completed, the appearances of the adhesive layers of the test samples were visually evaluated.

The results were evaluated in three levels, “very good” (“⊚” in [Table 1]), “good” (“◯” in [Table 1]), and “no good” (none in [Table 1]).

“Very good” indicates a state in which no change was observed in the appearance before the sterilization treatment.

“Good” indicates a state in which the appearance changed to an extent that small cracks were observed although they were not rendered unusable.

“No good” indicates a state in which degradation, for example, bubbles and cracks, was observed and the example was not usable.

### [Evaluation Results]

As shown in [Table 1], the evaluation results of Examples 1 to 7 were all “very good” but the evaluation results of Comparative Examples 1 to 5 were all “good.”

In this manner, in 300 hydrogen peroxide plasma sterilizations, in Comparative Examples 1 to 5 in which no inorganic zwitterion exchanger was included at all, there were no visually observable changes in the appearance. However, no change was observed in the appearances of all of the adhesive layers of Examples 1 to 7. It was found that the adhesive layers of Examples 1 to 7 had superior sterilizing gas resistance.

Here, as described above, the sterilizing gas resistance of all of the adhesive layers of Comparative Examples 3 to 5 in which the organic ion exchanger was included was inferior to that of the examples. However, comparing the changes in the appearance between the adhesive layers of Comparative Examples 3 to 5 in which the organic ion exchanger was included, the sterilizing gas resistance of the adhesive layer of Comparative Example 3 in which the organic zwitterion exchanger was included was better than the sterilizing gas resistance of the adhesive layers of Comparative Examples 4 and 5 in which no organic zwitterion exchanger was added.

Here, the reasons why the organic zwitterion exchanger in Comparative Example 3 had inferior sterilization resistance to the inorganic zwitterion exchanger in the examples were speculated.

One of the reasons why the organic zwitterion exchanger was inferior to the inorganic zwitterion exchanger was thought to be that the matrix of the organic zwitterion exchanger was an organic substance.

As a basic characteristic of the sterilizing gas, there is a characteristic of decomposing bacteria (organic substances) and performing sterilization. Since the matrix was an organic substance in the organic zwitterion exchanger, like bacteria, it was decomposed (or deteriorated) due to a sterilizing gas. On the other hand, it was thought that, since the matrix was an inorganic substance in the inorganic zwitterion exchanger, decomposition (or deterioration) due to a sterilizing gas was unlikely to occur.

Therefore, in the case of the organic zwitterion exchanger, even if anions and cations were captured, under a sterilizing gas, deterioration of the organic zwitterion exchanger itself was not negligible. Therefore, it was thought that, when the organic zwitterion exchanger was included, an evaluation result of favorable sterilizing gas resistance as in the case in which the inorganic zwitterion exchanger was included was not obtained.

In addition, it was thought that, since the inorganic zwitterion exchanger had superior compatibility with the epoxy resin compared to the organic zwitterion exchanger, a distance between an epoxy molecule and an ion exchanger particle was shorter in the inorganic zwitterion exchanger.

Therefore, it was thought that the inorganic zwitterion exchanger had a higher probability of blocking epoxy molecules from chemical attack of a sterilizing gas compared to the organic zwitterion exchanger.

### Examples Related to the Second Embodiment

Next, Examples 8 to 11 of the adhesive composition (II) of the second embodiment will be described together with Comparative Examples 6 and 7.

In the following [Table 4], compositions and evaluation results of the adhesive compositions of Examples 8 to 11 and Comparative Examples 6 and 7 are shown.

### [Table 4]

### Example 8

As shown in [Table 4], the main agent in the adhesive composition (II) of Example 8 was formed by mixing 9.4 parts by mass of a bisphenol A type epoxy resin (hereinafter referred to as an “epoxy resin α” sometimes), 6 parts by mass of a phenol novolak type epoxy resin (hereinafter referred to as an “epoxy resin β” sometimes), and 4 parts by mass of an acrylic rubber component. Specific materials of the bisphenol A type epoxy resin and the phenol novolak type epoxy resin were the same as the epoxy resins used in the main agent in Example 1.

As the curing agent in the adhesive composition (II) of Example 8, 10 parts by mass of the amine type curing agent was used. Specific materials of the amine type curing agent were the same as those in the amine type curing agent in Example 1.

As the filler in the adhesive composition (II) of Example 8, 70 parts by mass of alumina as an inorganic filler was used. Specifically, as alumina, Denka spherical alumina DAW-05 (product name; commercially available from Denka Company Ltd.) was used. The alumina was spherical particles having a specific gravity of 3.9 (density of 3.9 g/cm³), and an aspect ratio of 0 or more and less than 0.5.

About 45 parts by mass of the inorganic filler was included with respect to 10 parts by mass of the epoxy resin in the main agent.

As the inorganic zwitterion exchanger in the adhesive composition (II) of Example 8, 0.6 parts by mass of the inorganic zwitterion exchanger C (refer to [Table 3]) was used.

In the adhesive compositions (II) of Examples 9 to 11, the content of any of the main agent, the curing agent, and the filler, and a material of the filler were different from each other.

### Example 9

The composition of the main agent in Example 9 included 15 parts by mass of the epoxy resin α, 7 parts by mass of the epoxy resin β, and 1 part by mass of the acrylic robber component. In Example 9, 9 parts by mass of the curing agent was included. As the filler in Example 9, 57.3 parts by mass of zirconia as an inorganic filler was used. Specifically, zirconia bead DZBϕ7 was used. The zirconia was spherical particles having a specific gravity of 6.0 (density of 6.0 g/cm³), and an aspect ratio of 0 or more and less than 0.5.

About 26 parts by mass of the inorganic filler was included with respect to 10 parts by mass of the epoxy resin in the main agent.

### Example 10

The composition of the main agent in Example 10 included 17 parts by mass of the epoxy resin α, 9 parts by mass of the epoxy resin β, and 1 part by mass of the acrylic rubber component. In Example 10, 12 parts by mass of the curing agent was included.

As the filler in Example 10, 70 parts by mass of tungsten trioxide as an inorganic filler was used. Specifically, A2-WO3 (product name; commercially available from A.L.M.T. Corp.) was used. The tungsten trioxide was spherical particles having a specific gravity of 7.16 (density of 7.16 g/cm³), and an aspect ratio of 0 or more and less than 0.5.

About 27 parts by mass of the inorganic filler was included with respect to 10 parts by mass of the epoxy resin in the main agent.

### Example 11

The composition of the main agent in Example 11 included 12 parts by mass of the epoxy resin α, 6 parts by mass of the epoxy resin β, and 1 part by mass of the acrylic rubber component. In Example 11, 8 parts by mass of the curing agent was included.

As the filler in Example 11, 74 parts by mass of silicon nitride as an inorganic filler was used. Specifically, S-30 (product name; commercially available from MARUWA) was used. The silicon nitride was spherical particles having a specific gravity of 3.22 (density of 3.22 g/cm³), and an aspect ratio of 0 or more and less than 0.5.

The content of about 41 parts by mass of the inorganic filler was included with respect to 10 parts by mass of the epoxy resin in the main agent.

### Comparative Example 6

In the adhesive composition of Comparative Example 6, the epoxy resin α, the curing agent, and the type of filler were the same as those in Example 1. However, the epoxy resin β, the acrylic rubber component, and the inorganic zwitterion exchanger were not included at all.

In Comparative Example 6, 53 parts by mass of the epoxy resin α, 21 parts by mass of the curing agent, and 25 parts by mass of the filler were included.

Comparative Example 6 differed from the adhesive compositions (I) and (II) in which no inorganic zwitterion exchanger was included.

### Comparative Example 7

In the adhesive composition of Comparative Example 7, the epoxy resin α, the epoxy resin β, the acrylic component, and the type of curing agent were the same as those in Example 1. However, no inorganic zwitterion exchanger was included. As the filler of the adhesive composition of Comparative Example 7, silica was used. The silica was spherical particles having a specific gravity of 1.8 (density 1.8 g/cm³), and an aspect ratio of 0 or more and less than 0.5.

In Comparative Example 7, 37 parts by mass of the epoxy resin α, 17 parts by mass of the epoxy resin β, 2 parts by mass of the acrylic component, 22 parts by mass of the curing agent, and 22 parts by mass of the filler were included.

Comparative Example 7 differed from those of the adhesive compositions (I) and (II) in that no inorganic zwitterion exchanger was included.

### [Evaluation]

In the evaluation of Examples 8 to 11, and Comparative Examples 6 and 7, an acoustic impedance (shown as “acoustic IMP” in [Table 4]), an attenuation rate, a sterilizing gas resistance, and processability were evaluated.

In the evaluation of the acoustic impedance and the attenuation rate, as measurement samples, using the adhesive compositions of Examples 8 to 11, and Comparative Examples 6 and 7, cured resin layers in the form of 10 mm in length×30 mm in width×1 mm in thickness were produced. Ultrasonic transducers for measurement having the configuration of the second embodiment were produced using the cured resin layers.

As a method of measuring an acoustic impedance and an attenuation rate, a method according to a water immersion multiple reflection method in which no comparison measurement piece was used in a method of measuring an ultrasonic attenuation coefficient of a solid according to JIS Z 2354 was used. In this case, the ultrasonic transducer for measurement was driven al a frequency of 5 MHz.

When the acoustic impedance was greater than 3 MRayls and was 7 MRayls or less, it was evaluated as “good” (“◯” in [Table 4]), and when the acoustic impedance was 3 MRayls or less or greater than 7 MRayls, it was evaluated as “no good” (in “X” in [Table 4]).

Here, 1 MRayl is 1×10⁶ kg/(m²·s).

When the attenuation rate was greater than 3 dB/cm/MHz and was 4 dB/cm/MHz or less, it was evaluated as “good” (“◯” in [Table 4]), and when the attenuation rate was 3 dB/cm/MHz or less or greater than 4 dB/cm/MHz, it was evaluated as “no good” (“X” in [Table 4]).

The test of the sterilizing gas resistance was performed in the same manner as in the sterilization treatment in the examples related to the first embodiment except that the above ultrasonic transducer for measurement was used as a test sample. In addition, using the ultrasonic transducer for measurement before the resistance test started and after the resistance test ended, images of the same biotissues were acquired. The examples and the comparative examples were evaluated by observing changes in the image quality before and after the resistance test started.

When there was no change in the image quality, the sterilizing gas resistance was evaluated as “good” (“◯” in [Table 4]), and when there was change in the image quality, the sterilizing gas resistance was evaluated as “no good” (“X” in [Table 4]).

The processability was evaluated based on the flowability when the adhesive compositions were poured into a mold for forming the above cured resin layer, and particularly, based on whether molding was possible without trapping air.

When casting was possible without trapping air, the processability was evaluated as “good” (“◯” in [Table 4]), and when casting was not possible or casting was possible but air was trapped, the processability was evaluated as “no good” (“X” in [Table 4]).

### [Evaluation Results]

As shown in [Table 4], in the evaluation results of the ultrasonic transducers for measurement of Examples 8 to 11, all of the acoustic impedance, the attenuation rate, the sterilizing gas resistance, and the processability were evaluated as “good.” Therefore, the comprehensive evaluation was “good” (shown as “◯” in [Table 4]).

On the other hand, in the evaluation results of the ultrasonic transducer for measurement of Comparative Example 6, since the sterilizing gas resistance was evaluated as “no good,” the comprehensive evaluation was “no good” (shown as “X” in [Table 4]).

In Comparative Example 6, the reason why the evaluation result of the sterilizing gas resistance was “no good” was thought to be that, since no inorganic zwitterion exchanger was included in the cured resin layer, deterioration occurred due to chemical attack according to a sterilizing gas.

In the evaluation results of the ultrasonic transducer for measurement of Comparative Example 7, since the acoustic impedance, the attenuation rate, and the sterilizing gas resistance were evaluated as “no good,” the comprehensive evaluation was “no good.”

In Comparative Example 7, the reason why the evaluation results of the acoustic impedance and the attenuation rate were “no good” was thought to be that the specific gravity of silica included in the cured resin layer was smaller than the specific gravity of alumina, zirconia, tungsten trioxide, and silicon nitride. Although improvement by increasing the content of silica was conceivable, when the content of the silica increased, there is a risk of the formability deteriorating.

In Comparative Example 7, the reason why the evaluation result of the sterilizing gas resistance was “no good” was thought to be the same as in Comparative Example 6.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

For example, the adhesive composition of the first embodiment may be used for the ultrasonic transducer and the ultrasonic endoscope of the second embodiment.

For example, the adhesive composition of the second embodiment may be used for the endoscope of the first embodiment at a part other than the acoustic matching layer.

TABLE 1

| | Composition of adhesive composition | | | | | |
|---|---|---|---|---|---|---|
| | (parts by mass) | | | | | Evaluation of |
| | Main | Curing | | Ion | | sterilizing |
| | agent | agent | Filler | exchanger | Type of ion exchanger | gas resistance |
| Example 1 | 103 | 40 | 40 | 5 | Inorganic zwitterion exchanger A | ⊚ |
| Example 2 | 103 | 40 | 40 | 5 | Inorganic zwitterion exchanger B | ⊚ |
| Example 3 | 103 | 40 | 40 | 5 | Inorganic zwitterion exchanger C | ⊚ |
| Example 4 | 103 | 40 | 40 | 5 | Inorganic zwitterion exchanger D | ⊚ |
| Example 5 | 103 | 40 | 40 | 5 | Inorganic zwitterion exchanger E | ⊚ |
| Example 6 | 103 | 40 | 40 | 5 | Inorganic zwitterion exchanger F | ⊚ |
| Example 7 | 103 | 40 | 40 | 5 | Inorganic zwitterion exchanger G | ⊚ |
| Comparative | 103 | 40 | 40 | 5 | Inorganic cation exchanger a | ◯ |
| Example 1 | | | | | | |
| Comparative | 103 | 40 | 40 | 5 | Inorganic anion exchanger b | ◯ |
| Example 2 | | | | | | |
| Comparative | 103 | 40 | 40 | 5 | Organic zwitterion exchanger c | ◯ |
| Example 3 | | | | | | |
| Comparative | 103 | 40 | 40 | 5 | Organic cation exchanger d | ◯ |
| Example 4 | | | | | | |
| Comparative | 103 | 40 | 40 | 5 | Organic anion exchanger e | ◯ |
| Example 5 | | | | | | |

TABLE 2

| | Composition | | | |
|---|---|---|---|---|
| | (parts by mass) | Type | Product name | Manufacturer |
| Main agent | 10 | Bisphenol A type epoxy | Adeka resin EP | ADEKA |
| | | resin | (registered | |
| | | | trademark)-4100E | |
| | 3 | Bisphenol F type epoxy | Acryset (registered | Nippon Shokubai |
| | | resin + acrylic rubber | trademark) BPF307 | Co., Ltd. |
| | 60 | Bisphenol A type epoxy | Acryset (registered | Nippon Shokubai |
| | | resin + acrylic rubber | trademark) BPA328 | Co., Ltd. |
| | 30 | Phenol novolak type | jER (registered | Mitsubishi Chemical |
| | | epoxy resin | trademark) 152 | Corporation |
| Curing agent | 40 | Meta-xylylenediamine + | | Mitsubishi Gas |
| | | meta-xylylenediamine | | Chemical Company, Inc. |
| | | derivatives | | |
| Filler | 40 | Silica | EXR-3 (LV) | Tatsumori Ltd. |

TABLE 3

| | Component | Product name | Manufacturer |
|---|---|---|---|
| Inorganic | Sb- and | IXE (registered | Toagosei Co., Ltd. |
| zwitterion | Bi-based | trademark)-600 | |
| exchanger A | | | |
| Inorganic | Sb- and | IXE (registered | Toagosei Co., Ltd. |
| zwitterion | Bi-based | trademark)-633 | |
| exchanger B | | | |
| Inorganic | Zr- and | IXE (registered | Toagosei Co., Ltd. |
| zwitterion | Bi-based | trademark)-6107 | |
| exchanger C | | | |
| Inorganic | Zr- and | IXE (registered | Toagosei Co., Ltd. |
| zwitterion | Bi-based | trademark)-6136 | |
| exchanger D | | | |
| Inorganic | Zr-, Mg- and | IXEPLAS (registered | Toagosei Co., Ltd. |
| zwitterion | Al-based | trademark)-A1 | |
| exchanger E | | | |
| Inorganic | Zr-, Mg- and | IXEPLAS (registered | Toagosei Co., Ltd. |
| zwitterion | Al-based | trademark)-A2 | |
| exchanger F | | | |
| Inorganic | Zr- and | IXEPLAS (registered | Toagosei Co., Ltd. |
| zwitterion | Bi-based | trademark)-B1 | |
| exchanger G | | | |
| Inorganic | Zr-based | IXE (registered | Toagosei Co., Ltd. |
| cation | | trademark)-100 | |
| exchanger a | | | |
| Inorganic | Zr-based | IXE (registered | Toagosei Co., Ltd. |
| anion | | trademark)-800 | |
| exchanger b | | | |
| Organic | Crosslinked | Diaion (registered | Mitsubishi Chemical |
| zwitterion | polystyrene | trademark) AMP03 | Corporation |
| exchanger c | | | |
| Organic | Crosslinked | Diaion (registered | Mitsubishi Chemical |
| cation | polystyrene | trademark) PK208 | Corporation |
| exchanger d | | | |
| Organic | Crosslinked | Diaion (registered | Mitsubishi Chemical |
| anion | polystyrene | trademark) PA306S | Corporation |
| exchanger e | | | |

TABLE 4

| | Composition of adhesive composition (parts by mass) | | | | | | | | | | | |
|---|---|---|---|---|---|---|---|---|---|---|---|---|
| | Main agent | | | | | | | Evaluation results | | | | |
| | Bisphenol | Phenol | Acrylic | | | | | | | Steriliz- | | Compre- |
| | A type | novolak | rubber | | | Inorganic | | Acous- | Atten- | ing gas | | hensive |
| | epoxy | type epoxy | compo- | Curing | | zwitterion | | tic | uation | resis- | Process- | evalu- |
| | resin | resin | nent | agent | Filler | exchanger | Type of filler | IMP | rate | tance | ability | ation |
| Example 8 | 9.4 | 6 | 4 | 10 | 70 | 0.6 | Alumina | ◯ | ◯ | ◯ | ◯ | ◯ |
| Example 9 | 15 | 7 | 1 | 9 | 57.3 | 0.7 | Zirconia | ◯ | ◯ | ◯ | ◯ | ◯ |
| Example 10 | 17 | 9 | 1 | 12 | 70 | 1 | Tungsten trioxide | ◯ | ◯ | ◯ | ◯ | ◯ |
| Example 11 | 12 | 6 | 1 | 8 | 74 | 1 | Silicon nitride | ◯ | ◯ | ◯ | ◯ | ◯ |
| Comparative | 53 | 0 | 0 | 21 | 25 | 0 | Alumina | ◯ | ◯ | X | ◯ | X |
| Example 6 | | | | | | | | | | | | |
| Comparative | 37 | 17 | 2 | 22 | 22 | 0 | Silica | X | X | X | ◯ | X |
| Example 7 | | | | | | | | | | | | |

What is claimed is:

1. An adhesive composition comprising:
an epoxy resin as a main component; and
an inorganic zwitterion exchanger.

2. The adhesive composition according to claim 1,
wherein the inorganic zwitterion exchanger is an inorganic compound including at least one type of metal atom selected from a group consisting of bismuth, antimony, zirconium, magnesium, and aluminum.

3. The adhesive composition according to claim 1,
wherein 0.1 parts by mass or more and 1.0 part by mass or less of the inorganic zwitterion exchanger is added with respect to 10 parts by mass of the epoxy resin.

4. The adhesive composition according to claim 1,
wherein the epoxy resin includes at least one type of epoxy resin selected from a group consisting of a bisphenol A type epoxy resin, a bisphenol F type epoxy resin, and a phenol novolak type epoxy resin.

5. The adhesive composition according to claim 1, further comprising:
a curing agent including at least one selected from a group consisting of xylenediamine, a polyamine, a tertiary amine, and derivatives thereof.

6. The adhesive composition according to claim 1, further comprising:
an inorganic filler.

7. The adhesive composition according to claim 6,
wherein the inorganic filler includes at least one type of inorganic filler selected from a group consisting of alumina, zirconia, silicon nitride, silicon carbide, tungsten trioxide, diamond, sapphire, aluminum nitride, boron nitride, and magnesium oxide.

8. The adhesive composition according to claim 6,
wherein 30 parts by mass or more and 300 parts by mass or less of the inorganic filler is included with respect to 10 parts by mass of the epoxy resin.

9. The adhesive composition according to claim 6,
wherein the inorganic filler is spherical particles having an aspect ratio of 0 or more and less than 0.5.

10. An ultrasonic transducer, comprising:
an acoustic matching layer including a cured resin layer, the cured resin layer being obtained by curing the adhesive composition according to claim 6.

11. An endoscope, comprising:
two constituent members; and
an adhesive layer obtained by curing the adhesive composition according to claim 1,
wherein the two constituent members are bonded to each other by the adhesive layer.

12. An ultrasonic endoscope, comprising:
the ultrasonic transducer according to claim 10.
